(19) United States
(12) Patent Application Publication
(10) Pub. No.: US 2026/0325339 A1
(43) Pub. Date: Oct. 22, 2026

(54) FLUID COLLECTION DEVICES, SYSTEMS, AND METHODS

(71) Applicant: PUREWICK CORPORATION, Covington, GA (US)

(72) Inventor: Lyndsay Alison Sweeney, Decatur, GA (US)

(21) Appl. No.: 19/638,741

(22) Filed: Apr. 3, 2026

Related U.S. Application Data
(63) Continuation of application No. 17/051,600, filed on Apr. 29, 2019, now Pat. No. 12,622,803.
(60) Provisional application No. 62/665,711, filed on May 2, 2018.

(51) Int. Cl. A61F 5/455; A61F 5/44

(52) U.S. Cl. CPC A61F 5/455; A61F 5/4401

(57) ABSTRACT

Example fluid collection devices and methods of assembling the fluid collection devices. The fluid collection devices include a fluid impermeable barrier and a fluid permeable body. The fluid impermeable barrier at least partially defines a chamber and includes an opening extending therethrough. The opening is configured to be positioned adjacent to or receive therein a urethra of a subject. The fluid permeable body has a singular porous material in a substantially cylindrical shape and positioned at least partially within the chamber.

### CROSS-REFERENCE TO RELATED APPLICATIONS

This application is a continuation of U.S. application Ser. No. 17/051,600 filed on Oct. 29, 2020, which is a National Phase of International Application No. PCT/US2019/029616 filed on Apr. 29, 2019, which claims priority from U.S. Provisional Application No. 62/665,711 filed on May 2, 2018, the disclosure of each of which is incorporated herein, in its entirety, by this reference.

### BACKGROUND

An individual may have limited or impaired mobility such that typical urination processes are challenging or impossible. For example, the individual may have surgery or a disability that impairs mobility. In another example, the individual may have restricted travel conditions such as those experienced by pilots, drivers, and workers in hazardous areas. Additionally, fluid collection from the individual may be needed for monitoring purposes or clinical testing.

Bed pans and urinary catheters, such as a Foley catheter, can be used to address some of these circumstances. However, bed pans and urinary catheters have several problems associated therewith. For example, bed pans can be prone to discomfort, pressure ulcers spills, and other hygiene issues. Urinary catheters can be uncomfortable, painful, and can cause urinary tract infections.

Thus, users and manufacturers of fluid collection devices continue to seek new and improved devices, systems, and methods to collect urine.

### SUMMARY

Embodiments disclosed herein are fluid collection devices and methods of assembling fluid collection devices. In an embodiment, a fluid collection device includes a fluid impermeable barrier, a fluid permeable body, and a reservoir. The fluid impermeable barrier at least partially defines a chamber. The fluid impermeable barrier includes an opening extending therethrough. The opening is configured to be positioned adjacent to or receive therein a urethra of a subject. The fluid permeable body is a singular porous material in a substantially cylindrical shape and positioned at least partially within the chamber. The reservoir is at least partially defined by the fluid permeable body. The fluid permeable body is configured to wick fluid away from the opening to the reservoir.

In an embodiment, a method of assembling a fluid collection device is disclosed. The method includes providing a fluid impermeable barrier. The fluid impermeable barrier at least partially defines a chamber and has an opening extending therethrough. The opening is configured to be positioned adjacent to or receive therein a urethra of a subject. The method also includes inserting a substantially cylindrical and fluid permeable body into the chamber of the fluid impermeable barrier thereby forming a fluid collection device. The fluid permeable body at least partially defines a reservoir when the fluid permeable body is inserted into the chamber. The fluid permeable body includes a singular porous material that is substantially cylindrical in shape and configured to wick fluid away from the opening to the reservoir.

In an embodiment, a fluid collection device includes a fluid impermeable barrier, an opening, a fluid permeable layer, a reservoir, and a conduit. The fluid impermeable barrier at least partially defines a chamber. The opening extends into the chamber and is configured to be positioned adjacent to or receive therein a urethra of a subject. The fluid permeable layer is positioned within the chamber and includes a singular porous material configured to wick any fluid away from the opening. The reservoir is formed within the chamber and partially defined by a portion of the fluid permeable layer and an impermeable border. At least a portion of the singular porous material of the fluid permeable layer extends continuously between the opening and the reservoir to wick any fluid from the opening to the reservoir. The conduit includes an inlet and an outlet. The inlet extends to the reservoir and provides fluid communication between the reservoir and the outlet.

In an embodiment, a method to collect fluid includes positioning a fluid permeable body of a fluid collection device adjacent to a urethra of a subject. The fluid permeable body is disposed within a chamber of a fluid impermeable barrier of the fluid collection device and exposed to the urethra of the subject through an opening in the fluid collection device. The method also includes securing the fluid collection device to the user. The method also includes receiving fluids from the female urethra into the chamber of the fluid collection device.

In an embodiment, a fluid collection device includes a body having an opening and a closed distal end. The body includes a fluid impermeable side wall at least partially defining a chamber within the body. The body also includes a fluid permeable layer positioned with the chamber to partially define a reservoir at the distal end, the fluid permeable layer including a singular porous material. The body is configured to be disposed with a urethra of a subject disposed within the opening or adjacent to the opening. The body also is configured to receive urine discharged from the urethra in the opening, the fluid permeable layer is configured to wick urine discharged from the urethra away from the subject to the reservoir to have the urine withdrawn from the reservoir via a conduit.

Features from any of the disclosed embodiments may be used in combination with one another, without limitation. In addition, other features and advantages of the present disclosure will become apparent to those of ordinary skill in the art through consideration of the following detailed description and the accompanying drawings.

### BRIEF DESCRIPTION OF THE DRAWINGS

The drawings illustrate several embodiments of the present disclosure, wherein identical reference numerals refer to identical or similar elements or features in different views or embodiments shown in the drawings.

FIG. 1A is an isometric view of a female fluid collection device, according to an embodiment.

FIG. 1B is a front view of a female fluid collection device worn on a female user, according to an embodiment.

FIG. 1C is an exploded view of the female fluid collection device of FIG. 1A, according to an embodiment.

FIGS. 2A and 2B are cross-sectional views of the female fluid collection device of FIG. 1 taken along line 2-2 thereof, according to embodiments.

FIG. 3 is a top view of a fluid permeable body, according to an embodiment.

FIG. 4 is a flow diagram of a method of assembling a fluid collection device, according to an embodiment.

FIG. 5A is an isometric view of a urine collecting assembly, according to an embodiment.

FIG. 5B is a schematic cross-sectional view of a urine collecting assembly, according to an embodiment.

FIG. 5C is an exploded view of the urine collecting assembly of FIG. 5A, according to an embodiment.

FIG. 6 is a flow diagram of a method to collect fluid, according to an embodiment.

### DETAILED DESCRIPTION

Embodiments disclosed herein are fluid collection devices and methods of assembling fluid collection devices. In an embodiment, a fluid collection device includes a fluid impermeable barrier, a fluid permeable body, and a reservoir. The fluid impermeable barrier at least partially defines a chamber. The fluid impermeable barrier includes an opening extending therethrough. The opening is configured to be positioned adjacent to or receive therein a urethra of a subject. The fluid permeable body is a singular porous material in a substantially cylindrical shape and positioned at least partially within the chamber. The reservoir is at least partially defined by the fluid permeable body. The fluid permeable body is configured to wick fluid away from the opening to the reservoir.

As noted above, in many embodiments disclosed herein, the fluid permeable body includes a singular and porous body. That is, during use, the fluid permeable body extends from a conduit or elongated opening to interface the fluid impermeable barrier and the opening. A singular fluid permeable body can reduce the number of components in the fluid collection device, reduces the assembly time of the fluid collection device, requires shelf-life data for only a single component, and/or provides a latex-free single component.

The fluid collection devices disclosed herein are configured to collect fluids from an individual. The fluids collected by the fluid collection devices can include urine. The fluids collected by the fluid collection devices can also include at least one of vaginal discharge, penile discharge, reproductive fluids, blood, sweat, or other bodily fluids.

Fluid collection devices described herein may be used in fluid collection systems. The fluid collection systems can include a fluid collection device, a fluid storage container, and a portable vacuum source. Fluid (e.g., urine or other bodily fluids) collected in the fluid collection device may be removed from the fluid collection device via a conduit which protrudes into an interior region of the fluid collection device. For example, a first open end of the conduit may extend into the fluid collection device to a reservoir therein. The second open end of the conduit may extend into the fluid collection device or the portable vacuum source. The suction force may be introduced into the interior region of the fluid collection device via the first open end of the conduit responsive to a suction (e.g., vacuum) force applied at the second end of the conduit. The suction force may be applied to the second open end of the conduit by the portable vacuum source either directly or indirectly.

Fluid collection devices describe herein may be shaped and sized to be positioned adjacent to a female urethra or have a male urethra positioned therethrough (e.g., receive a penis therein). For example, the fluid collection device may include a fluid impermeable barrier at least partially defining a chamber (e.g., interior region of the fluid collection device) of the fluid collection device. The fluid impermeable barrier also defines an opening extending therethrough from the external environment. The opening may be positioned adjacent to a female urethra or have a male urethra positioned therethrough. The fluid collection device may include a fluid permeable body disposed within the fluid impermeable barrier. The conduit may extend into the fluid collection device at a first end region, through one or more of the fluid impermeable barrier, fluid permeable body to a second end region of the fluid collection device. Exemplary fluid collection devices for use with the systems and methods herein are described in more detail below.

In some embodiments, the portable vacuum source may be disposed in or on the fluid collection device. In such embodiments, the conduit may extend from the fluid collection device and attach to the portable vacuum source at a first point therein. An additional conduit may attach to the portable vacuum source at a second point thereon and may extend out of the fluid collection device, and may attach to the fluid storage container. Accordingly, a vacuum (e.g., suction) may be drawn through fluid collection device via the fluid storage container. Fluid, such as urine, may be drained from the fluid collection device using the portable vacuum source.

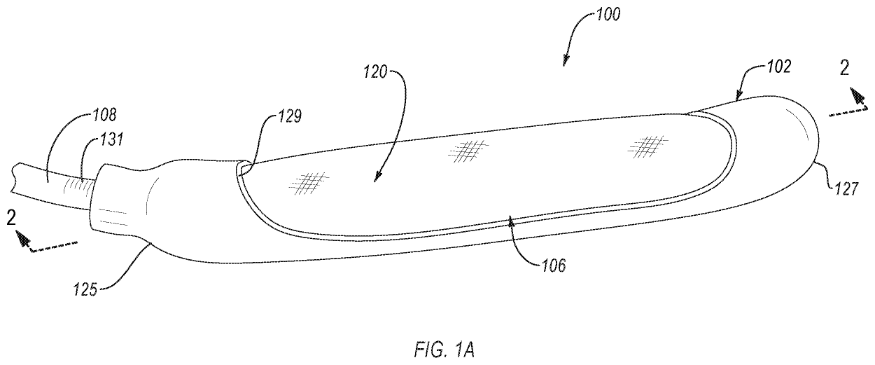

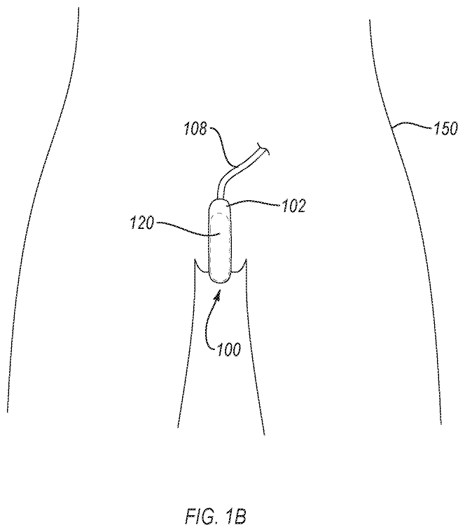

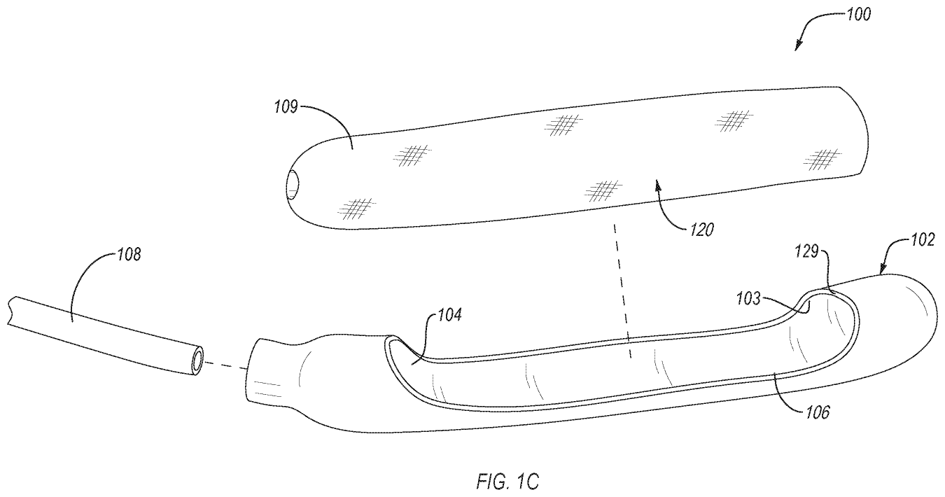

FIG. 1A is a perspective view of a fluid collection device 100, according to an embodiment. The fluid collection device 100 is an example of a female fluid collection device 100 that is configured to receive fluids from a female. The fluid collection device 100 includes a fluid impermeable barrier 102 having a first end region 125 and a second end region 127. The fluid impermeable barrier 102 at least partially defines a chamber 104 (e.g., interior region, shown in FIG. 1C) and includes an inward border or edge 129 defining an opening 106. The fluid impermeable barrier 102 is substantially cylindrical in shape between the first end region 125 and the second end region 127. The opening 106 is formed in and extends through the fluid impermeable barrier 102, thereby enabling fluids to enter the chamber 104 from outside of the fluid collection device 100. The opening 106 can be configured to be positioned adjacent to a female urethra.

The fluid collection device 100 may be positioned proximate to the female urethra and urine may enter the interior region of the fluid collection device 100 via the opening 106. The fluid collection device 100 is configured to receive the fluids into the chamber 104 via the opening 106. For example, the opening 106 can exhibit an elongated shape that is configured to extend from a first location below the urethral opening (e.g., at or near the anus or the vaginal opening) to a second location above the urethral opening (e.g., at or near the clitoris or the pubic hair). The opening 106 can exhibit an elongated shape since the space between the legs of a female is relatively small when the legs of the female are closed, thereby only permitting the flow of the fluids along a path that corresponds to the elongated shape of the opening 106. The opening 106 in the fluid impermeable barrier 102 can exhibit a width that is measured transverse to the longitudinal direction and may be at least about 10% of the circumference of the fluid collection device 100, such as about 25% to about 50%, about 40% to about 60%, about 50% to about 75%, about 65% to about 85%, or about 75% to about 100% of the circumference of the fluid collection device 100. The opening 106 can exhibit a width that is greater than 50% of the circumference of the fluid collection device 100 since the vacuum (e.g., suction) through the conduit 108 pulls the fluid into the conduit 108. In some embodiments, the opening 106 may be vertically oriented (e.g., having a major axis parallel to the longitudinal axis of the device 100). In some embodiments, (not shown), the opening 106 may be horizontally oriented (e.g., having a major axis perpendicular to the longitudinal axis of the device 100). In some embodiments, the inward border or edge 129 of the fluid impermeable barrier 102 defines the opening 106. The edge 129 can include two opposing arced portions, the arcs following the outer circumference or periphery of the substantially cylindrical fluid impermeable barrier 102. In an embodiment, the fluid impermeable barrier 102 can be configured to be attached to the individual, such as adhesively attached (e.g., with a hydrogel adhesive) to the individual. According to an embodiment, a suitable adhesive is a hydrogel layer, such as those disclosed in U.S. Patent Application Publication No. 2017/0189225, the disclosure of which is incorporated herein by reference in its entirety.

The fluid impermeable barrier 102 may also temporarily store the fluids in the chamber 104. For example, the fluid impermeable barrier 102 can be formed of any suitable fluid impermeable materials, such as a fluid impermeable polymer (e.g., silicone, polypropylene, polyethylene, polyethylene terephthalate, a polycarbonate, etc.), a metal film, another suitable material, or combinations thereof. As such, the fluid impermeable barrier 102 substantially prevents the fluids from exiting the portions of the chamber 104 that are spaced from the opening 106. The fluid impermeable barrier 102 is flexible, allowing the fluid collection device 100 to bend or curve when positioned against the body of a wearer.

The fluid collection device 100 can include a fluid permeable body 120 or layer disposed in the chamber 104. The fluid permeable body 120 can cover at least a portion (e.g., all) of the opening 106. The fluid permeable body 120 can be configured to wick any fluid away from the opening 106, thereby preventing the fluid from escaping the chamber 104. The fluid permeable body 120 also can wick the fluid generally towards an interior of the chamber 104, as discussed in more detail below. A portion of the fluid permeable body 120 can define a portion of an outer surface of the fluid collection device 100. Specifically, the portion of the fluid permeable body 120 defining the portion of the outer surface of the fluid collection device 100 can be the portion of the fluid permeable body 120 exposed by the opening 106 defined by the fluid impermeable barrier 102 that contacts the user. Moreover, the portion of the fluid permeable device defining the portion of the outer surface of the fluid collection device 100 may free from coverage by gauze or other wicking material at the opening.

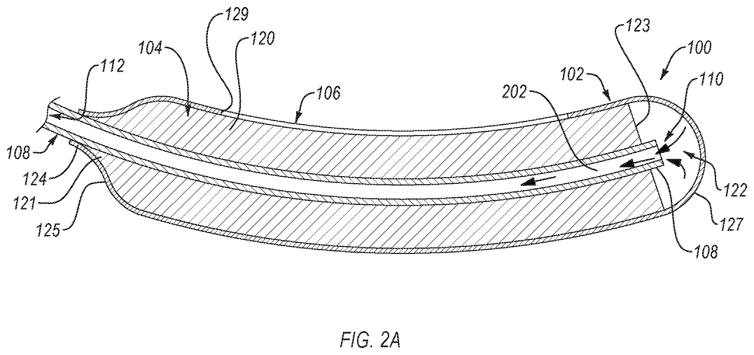

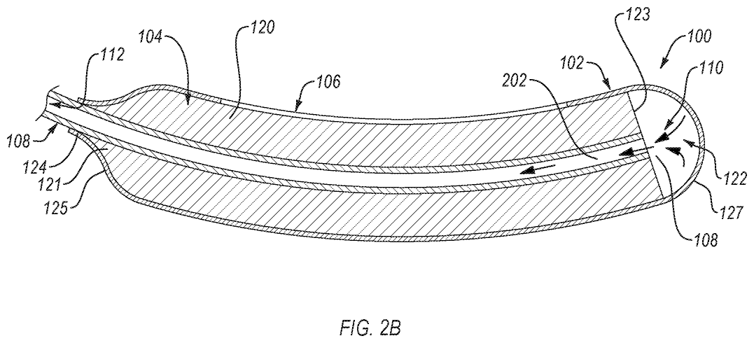

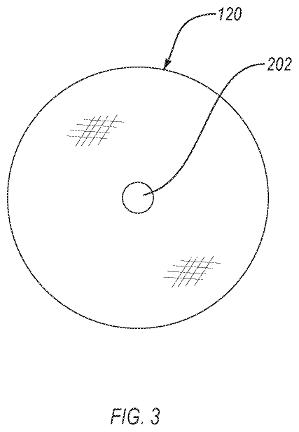

The fluid permeable body 120 can include any material that can wick the fluid. The permeable properties referred to herein can be wicking, capillary action, diffusion, or other similar properties or processes, and are referred to herein as “permeable” and/or “wicking.” Such “wicking” may exclude absorption into the wicking material. The fluid permeable body 120 can include a one-way fluid movement fabric. As such, the fluid permeable body 120 can remove fluid from the area around the female urethra, thereby leaving the urethra dry. The fluid permeable body 120 can enable the fluid to flow generally towards a reservoir 122 (shown in FIGS. 2A and 2B) of void space formed within the chamber 104. For example, the fluid permeable body 120 can include a porous or fibrous material, such as hydrophilic polyolefin. In some embodiments, the fluid permeable body 120 consists of or consists essentially of a porous or fibrous material, such as hydrophilic polyolefin. Examples of polyolefin that can be used in the fluid permeable body 120 include, but are not limited to, polyethylene, polypropylene, polyisobutylene, ethylene propylene rubber, ethylene propylene diene monomer, or combinations thereof. The porous or fibrous material can be extruded into a substantially cylindrically shape to fit within the chamber 104 of the fluid impermeable barrier 102. The fluid permeable body 120 can include varying densities or dimensions. Moreover, the fluid permeable body 120 can be manufactured according to various manufacturing methods, such as molding, extrusion, or sintering.

In some embodiments, the fluid permeable body 120 includes a singular and porous body. That is, during use, the fluid permeable body 120 extends from the conduit 108 to interface the fluid impermeable barrier 102 and the opening 106. In some embodiments, a majority of the outer surface 109 (shown in FIG. 1C) of the fluid permeable body 120 interfaces with an inner surface 103 (shown in FIG. 1C) of the fluid impermeable barrier 106. A singular fluid permeable body 120 is advantageous to conventional systems, which typically require an air-laid nonwoven pad covered by a ribbed fabric compression bandage, because a singular fluid permeable body 120 reduced the number of components in the fluid collection device 100, reduces the assembly time of the fluid collection device 100, requires shelf-life data for only a single component, and provides a latex-free single component. In some embodiments, at least a portion of the singular porous material of the fluid permeable body 120 extends continuously between the opening 106 and the reservoir 122 to wick any fluid from the opening 106 directly to the reservoir 122. Moreover, as the fluid impermeable barrier is flexible and the fluid permeable body 120 is configured to wick fluid from the body rather than absorb fluid from the body and hold the fluid against the body, the fluid collection device 100, in some embodiments, is free from a seal or cushioning ring on the inward edge 129 defining the opening 106.

FIG. 1B is a front view of a fluid collection device 100 in use on a female user 150. In use, the fluid permeable body 120 of the fluid collection device is positioned adjacent to a urethra of the user 150. The fluid permeable body 120 is disposed within a chamber 104 (shown in FIGS. 2A and 2B) of the fluid impermeable barrier 102 of the fluid collection device 100 and is exposed to the urethra of the user 150 through the opening 106 in the fluid collection device 100. The fluid collection device 100 can be secured to the user with any of a number of securing devices. Fluids received in the chamber 104 of the fluid collection device 100 from the urethra can be removed through the conduit 108.

FIG. 2 is a cross-sectional view of the fluid collection device 100 taken along line 2-2 of FIG. 1. The fluid collection device 100 also includes conduit 108 that is at least partially disposed in the chamber 104. The conduit 108 (e.g., a tube) includes an inlet 110 at a second end region 127 of the fluid impermeable barrier 102 and an outlet 112 at a first end region 125 of the fluid impermeable barrier 102 positioned downstream from the inlet 110. The conduit 108 provides fluid communication between an interior region of the chamber 104 and a fluid storage container (not shown) or a portable vacuum source (not shown). For example, the conduit 108 may directly or indirectly fluidly couple the interior region of the chamber 104 and/or the reservoir 122 with the fluid storage container or the portable vacuum source.

In the illustrated embodiment, the fluid permeable body 120 defines a bore 202 extending through the fluid permeable body 120 from a first body end 121 of the fluid permeable body 120 to a second body end 123 of the fluid permeable body 120 distal to the first body end 120. In other embodiments, the bore 202 extends only partially into the fluid permeable body from the first body end 121 of the fluid permeable body 120.

In the illustrated embodiment, the conduit 108 is at least partially disposed in the chamber 104 and interfaces at least a portion of the bore 202 of the fluid permeable body 120. For example, the conduit 108 may extend into the fluid impermeable barrier 102 from the first end region 125 (e.g., proximate to the outlet 112) and may extend through the bore 202 to the second end region 127 (e.g., opposite the first end region 125) to a point proximate to a reservoir 122 such that the inlet 110 is in fluid communication with the reservoir 122. For example, in the illustrated embodiment, the inlet 110 is positioned in the reservoir 122. However, in other embodiments, the inlet 110 may be positioned flush with or behind an end of the fluid permeable body 120 that partially defines the reservoir 122. The fluid collected in the fluid collection device 100 may be removed from the interior region of the chamber 104 via the conduit 108. The conduit 108 may include a flexible material such as plastic tubing (e.g., medical tubing). Such plastic tubing may include a thermoplastic elastomer, polyvinyl chloride, ethylene vinyl acetate, polytetrafluoroethylene, etc., tubing. In some embodiments, the conduit 108 may include silicone or latex.

The fluid impermeable barrier 102 can store fluids in a reservoir 122 therein. The reservoir 122 is an unoccupied portion of the chamber 104 and is void of other material. In some embodiments, the reservoir 122 is defined at least partially by the fluid permeable body 120 and the fluid impermeable barrier 102. The reservoir 122 may be disposed in any portion of the interior region of the chamber 104. For example, the fluid reservoir 122 may be positioned in the second end region 127 of the chamber 104. In the illustrated embodiment, the reservoir 122 is defined by the second body end 123 of the fluid permeable body 120 and the second end region 127 of the fluid impermeable barrier 122.

In an embodiment, the fluid impermeable barrier 102 can be air permeable and fluid impermeable. In such an embodiment, the fluid impermeable barrier 102 can be formed of a hydrophobic material that defines a plurality of pores. In an embodiment, one or more portions of at least the outer surface of the fluid impermeable barrier 102 can be formed from a soft and/or smooth material, thereby reducing chaffing. The fluid impermeable barrier 102 may include markings thereon, such as one or more markings to aid a user in aligning the device 100 on the wearer. For example, a line on the fluid impermeable barrier 102 (e.g., opposite the opening 106) may allow a healthcare professional to align the opening 106 over the urethra of the wearer. In examples, the markings may include one or more of alignment guide or an orientation indicator, such as a stripe or hashes. Such markings may be positioned to align the device 100 to one or more anatomical features such as a pubic bone, etc.

In an embodiment, the fluid permeable body 120 and at least a portion of the conduit 108 can at least substantially completely fill the chamber 104. In another example, the fluid permeable body 120 and at least a portion of the conduit may not substantially completely fill the chamber 104. In such an example, the fluid collection device 100 includes the reservoir 122 disposed in the chamber 104. The reservoir 122 is a substantially unoccupied portion of the chamber 104. The fluids that are in the chamber 104 can flow through the fluid permeable body 120 to the reservoir 122. The reservoir 122 can store at least some of the fluids therein. In these and other embodiments, the fluid permeable body 120, at least a portion of the conduit 108, and the reservoir 122 can at least substantially completely fill the chamber 104.

In an embodiment, the reservoir 122 can be located at the portion of the chamber 104 that is closest to the inlet 110 (e.g., the second end region). However, the reservoir 122 can be located at different locations in the chamber 104. For example, the reservoir 122 can be located at the end of the chamber 104 that is closest to the outlet 112. In these and other embodiments, the conduit 108 may extend through the fluid impermeable barrier to the reservoir 122 without extending through the fluid permeable body 120. Accordingly, in these and other embodiments, the fluid permeable body 120 may be free from the bore. In another embodiment, the fluid collection device 100 can include multiple reservoirs, such as a first reservoir that is located at the portion of the chamber of the chamber 104 that is closest to the inlet 110 (e.g., second end region) and a second reservoir that is located at the portion of the of the chamber 104 that is closest to the outlet 112 (e.g., first end region). In another example, the fluid permeable body 120 is spaced from at least a portion of the conduit 108 and the reservoir 122 can be the space between the fluid permeable body 120 and the conduit 108.

Other embodiments of fluid impermeable barriers, fluid permeable membranes, fluid permeable bodies, chambers, and their shapes and configurations are disclosed in U.S. patent application Ser. Nos. 15/612,325 filed on Jun. 2, 2017; 15/260,103 filed on Sep. 8, 2016; and 15/611,587 filed on Jun. 1, 2017, the disclosure of each of which is incorporated herein, in its entirety, by this reference.

The fluid impermeable barrier 102, the fluid permeable body 120 can be configured to have the conduit 108 at least partially disposed in the chamber 104. For example, the fluid permeable body 120 can be configured to form a space that accommodates the conduit 108, such as the bore 202. In another example, the fluid impermeable barrier 102 can define an aperture 124 sized to receive the conduit 108 (e.g., at least one tube). The at least one conduit 108 can be disposed in the chamber 104 via the aperture 124. The apertures 124 can be configured to form an at least substantially fluid tight seal against the conduit 108 or the at least one tube thereby substantially preventing the fluids from escaping the chamber 104.

When secured to the fluid collection device 100, the conduit 108 is configured to provide fluid communication with and at least partially extend between one or more of a fluid storage container (not shown) and a portable vacuum source (not shown). For example, the conduit 108 may be configured to be fluidly coupled to and at least partially extend between one or more of the fluid storage containers and the portable vacuum source. In an embodiment, the conduit 108 is configured to be directly connected to the portable vacuum source (not shown). In such an example, the conduit 108 can extend from the fluid impermeable barrier 102 by at least one foot, at least two feet, at least three feet, or at least six feet. In another example, the conduit 108 is configured to be indirectly connected to at least one of the fluid storage container (not shown) or the portable vacuum source (not shown). In some examples, the conduit may be frosted or opaque (e.g., black) to obscure visibility of the fluids therein. In some embodiments, the conduit is secured to a wearer's skin with a catheter securement device, such as a STATLOCK® catheter securement device available from C. R. Bard, Inc., including but not limited to those disclosed in U.S. Pat. Nos. 6,117,163; 6,123,398; and 8,211,063, the disclosures of which are all incorporated herein by reference in their entirety.

The inlet 110 and the outlet 112 are configured to provide fluid communication (e.g., directly or indirectly) between the portable vacuum source (not shown) and the chamber 104 (e.g., the reservoir 122). For example, the inlet 110 and the outlet 112 of the conduit 108 may be configured to directly or indirectly fluidly couple the portable vacuum source to the reservoir 122. In an embodiment, the inlet 110 and/or the outlet 112 can form a male connector. In another example, the inlet 110 and/or the outlet 112 can form a female connector. In an embodiment, the inlet 110 and/or the outlet 112 can include ribs that are configured to facilitate secure couplings. In an embodiment, the inlet 110 and/or the outlet 112 can form a tapered shape. In an embodiment, the inlet 110 and/or the outlet 112 can include a rigid or flexible material.

Locating the inlet 110 at or near a gravimetrically low point of the chamber 104 enables the conduit to receive more of the fluids than if inlet 110 was located elsewhere and reduce the likelihood of pooling (e.g., pooling of the fluids can cause microbe growth and foul odors). For instance, the fluids in the fluid permeable body 120 can flow in any direction due to capillary forces. However, the fluids may exhibit a preference to flow in the direction of gravity, especially when at least a portion of the fluid permeable body 120 is saturated with the fluids.

As the portable vacuum source applies a vacuum/suction in the conduit 108, the fluid(s) in the chamber 104 (e.g., such as in the reservoir 122 positioned at the first end region 125, the second end region 127, or other intermediary positions within the chamber 104) may be drawn into the inlet 110 and out of the fluid collection device 100 via the conduit 108.

In an embodiment, the conduit 108 is configured to be at least insertable into the chamber 104. In such an embodiment, the conduit 108 can include one or more markers 131 (shown in FIG. 1) on an exterior thereof that are configured to facilitate insertion of the conduit 108 into the chamber 104. For example, the conduit 108 can include one or more markings thereon that are configured to prevent over or under insertion of the conduit 108, such as when the conduit 108 defines an inlet 110 that is configured to be disposed in or adjacent to the reservoir 122. In another embodiment, the conduit 108 can include one or more markings thereon that are configured to facilitate correct rotation of the conduit 108 relative to the chamber 104. In an embodiment, the one or more markings can include a line, a dot, a sticker, or any other suitable marking. In examples, the conduit 108 may extend into the fluid impermeable barrier 102 from the first end region (e.g., proximate to the outlet 112) and may extend to the second end region (e.g., opposite the first end region) to a point proximate to the reservoir 122 such that the inlet 110 is in fluid communication with the reservoir 122. In some embodiments (not shown), the conduit 108 may enter the second end region and the inlet 110 may be disposed in the second end region (e.g., in the reservoir 122). The fluid collected in the fluid collection device 100 may be removed from the interior region of the chamber 104 via the conduit 108. The conduit 108 may include a flexible material such as plastic tubing (e.g., medical tubing) as disclosed herein. In some examples, the conduit 108 may include one or more portions that are resilient, such as to by having one or more of a diameter or wall thickness that allows the conduit to be flexible.

In an embodiment, one or more components of the fluid collection device 100 can include an antimicrobial material, such as an antibacterial material where the fluid collection device may contact the wearer or the bodily fluid of the wearer. The antimicrobial material can include an antimicrobial coating, such as a nitrofurazone or silver coating. The antimicrobial material can inhibit microbial growth, such as microbial growth due to pooling or stagnation of the fluids. In an embodiment, one or more components of the fluid collection device 100 (e.g., impermeable barrier 102, conduit 108, etc.) can include an odor blocking or absorbing material such as a cyclodextrine containing material or a thermoplastic elastomer (TPE) polymer.

In any of the embodiments disclosed herein the conduits 108 may include or be operably coupled to a flow meter (not shown) to measure the flow of fluids therein, one or more securement devices (e.g., a StatLock securement device, not shown) or fittings to secure the conduit 108 to one or more components of the systems or devices disclosed herein (e.g., portable vacuum source or fluid storage container), or one or more valves to control the flow of fluids in the systems and devices herein.

In an embodiment, at least one of portion of the conduit 108 of the fluid collection devices or systems herein can be formed of an at least partially opaque material which can obscure the fluids that are present therein. For example, the B section of the conduits 108 disclosed herein may be formed of an opaque material or translucent material while the A section may be formed of a transparent material or translucent material. In some embodiments, the B section may include transparent or translucent material. Unlike the opaque or nearly opaque material, the translucent material allows a user of the devices and systems herein to visually identify fluids or issues that are inhibiting the flow of fluids within the conduit 108.

In any of the examples, systems or devices disclosed herein, the system of fluid collection device may include moisture sensors (not shown) disposed inside of the chamber of the fluid collection device. In such examples, the moisture sensor may be operably coupled to a controller or directly to the portable vacuum source, and may provide electrical signals indicating that moisture is or is not detected in one or more portions of the chamber. The moisture sensor(s) may provide an indication that moisture is present, and responsive thereto, the controller or portable vacuum device may direct the initiation of suction to the chamber to remove the fluid therefrom. Suitable moisture sensors may include capacitance sensors, volumetric sensors, potential sensors, resistance sensors, frequency domain reflectometry sensors, time domain reflectometry sensors, or any other suitable moisture sensor. In practice, the moisture sensors may detect moisture in the chamber and may provide a signal to the controller or portable vacuum source to activate the portable suction device.

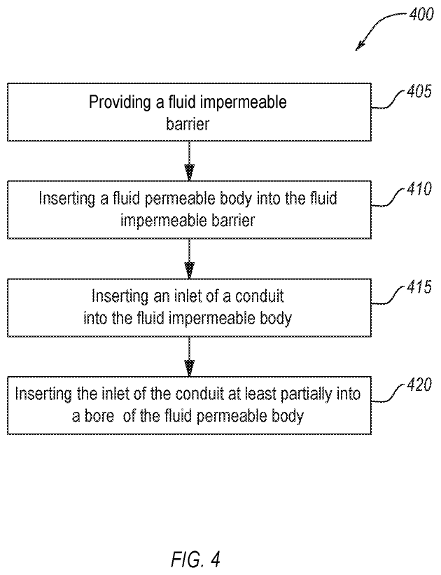

FIG. 4 is a flow diagram of a method 400 of assembling the fluid collection devices and/or fluid collection systems disclosed herein, according to an embodiment. The method 400 can include act 405, which recites providing a fluid impermeable barrier. The fluid impermeable barrier at least partially defines a chamber and also an opening extending therethrough. The opening is configured to be positioned adjacent to a female urethra or have a male urethra positioned therethrough. The fluid permeable body can include a singular porous hydrophilic polyolefin material extruded, molded, or sintered to a substantially cylindrical shape

The method can include act 410, which recites inserting a substantially cylindrical and fluid permeable body into the chamber of the fluid impermeable barrier. When the fluid permeable body is inserted into the chamber of the fluid impermeable barrier, the fluid permeable body interfaces at least a portion of the fluid impermeable barrier and covers at least a portion of the opening. The fluid permeable body includes a singular porous material that is substantially cylindrical in shape and configured to wick any fluid away from the opening. In some embodiments, act 410 can include inserting the fluid permeable body into the chamber of the fluid impermeable barrier such that a reservoir is defined within the chamber by a second body end of the fluid permeable body distal to the first body end and a second end region of the fluid impermeable barrier distal to the aperture. In some embodiments, act 410 can include inserting the substantially cylindrical and fluid permeable body into the chamber of the fluid impermeable barrier such that the fluid permeable body and the conduit fill substantially all of the chamber.

The method can include act 415, which recites inserting an inlet of a conduit into the fluid impermeable body. The conduit can be inserted into the fluid impermeable body through an aperture defined by the fluid impermeable barrier at a first end region of the fluid impermeable barrier. In some embodiments, act 415 can include inserting the inlet of the conduit into the bore at the first body end, through the bore of the fluid permeable body, through the second body end of the fluid permeable body, and into the reservoir such that the conduit extends from the reservoir, through the fluid permeable body, through the aperture to outside the fluid impermeable barrier.

The method can include an act 420, which recites inserting the inlet of the conduit at least partially into a bore at a first body end of the fluid permeable body. The bore extends at least partially through the fluid permeable body and is defined by the fluid permeable body. The conduit interfaces at least a portion of the fluid permeable body.

Acts 405, 410, 415, and 420 of the method 400 are for illustrative purposes. For example, the act 405, 410, 415, and 420 of the method 400 can be performed in different orders, split into multiple acts, modified, supplemented, or combined. In an embodiment, one or more of the acts 405, 410, 415, and 420 of the method 400 can be omitted from the method 400. Any of the acts 405, 410, 415, and 420 can include using any of the fluid collection devices or systems disclosed herein.

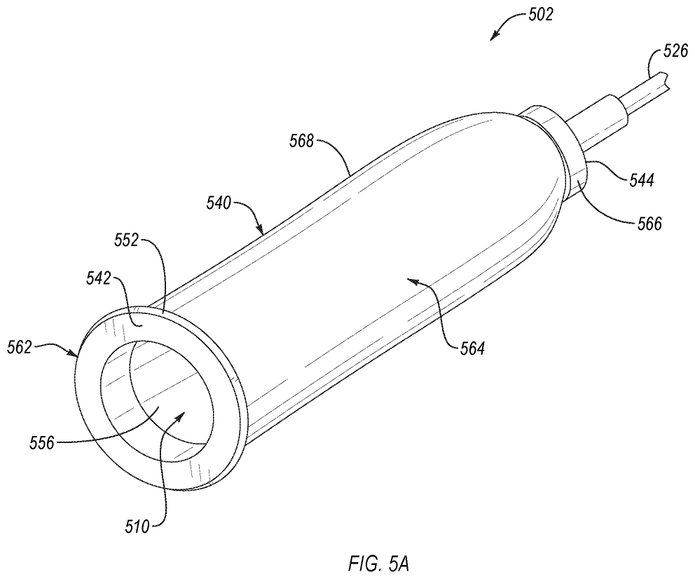

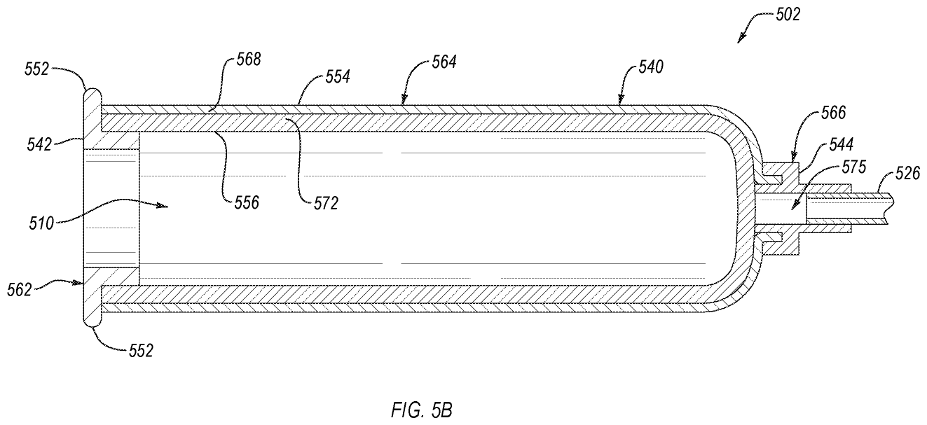

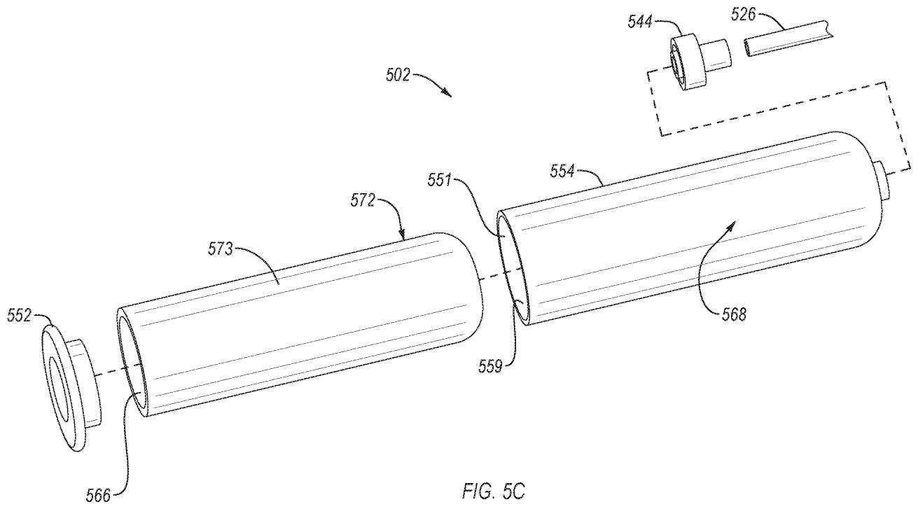

Also disclosed herein is a fluid collection device 502 configured for use with a male user. FIGS. 5A and 5B are an isometric view and a schematic cross-sectional view, respectively, of a fluid collection device 502 (or urine collecting assembly), according to an embodiment. FIG. 5C is an exploded view of the fluid collection device 502. The fluid collection device 502 can include a body 540 having a first end 542 and a second end 544, an opening 510 at a first end of the body, a reservoir 575 at the second end 544 of the body 544, and tubing 526 (or conduit) providing fluid communication with the reservoir 575. The first end 542 may include an open proximal end and the second end 544 may include closed distal end.

The body 540 of the fluid collection device 502 can include a ring 562 at or near the first end 542 of the body 540, a sheath 564 extending from or near the first end 542 to or near the second end 544 of the body 540, and a sump 566 at the second end 544 of the body 540. The sheath 564 is configured to prevent a fluid (e.g., urine) escaping from the opening 510 and to move the fluid towards the reservoir 575 in the sump 566 and the tubing 526. As such, referring to FIG. 5B, the sheath 564 can include a plurality of layers that facilitate the operation of the sheath 564. For example, the sheath 564 can include a fluid impermeable layer 568, and a fluid permeable layer 572. The fluid impermeable layer 568 can form an external surface 554 of the body 540 and prevent the fluid from leaking through the sheath 564. The fluid permeable layer 572 can form an internal surface 556 of the body 540 at least partially defining the opening 510 within the sheath 564. The fluid impermeable layer 568 may be substantially cylindrical in shape.

The fluid permeable layer 572 can be configured to substantially prevent the fluid that is in the fluid permeable layer 572 from flowing back into the opening 510. As such, fluid permeable layer 572 can remove fluid from around a penis thereby leaving the penis dry. The fluid permeable layer 572 can include a one-way fluid movement fabric. The fluid permeable layer 572 can enable the fluid to flow generally towards the tubing 526. The fluid permeable layer 572 can include any material that can wick the fluid. For example, the fluid permeable layer 572 can include a porous or fibrous material, such as hydrophilic polyolefin. In some embodiments, the fluid permeable layer 572 consists of or consists essentially of a porous or fibrous material, such as hydrophilic polyolefin. Examples of polyolefin that can be used in the fluid permeable layer 572 include, but are not limited to, polyethylene, polypropylene, polyisobutylene, ethylene propylene rubber, ethylene propylene diene monomer, or combinations thereof. The fluid permeable layer 572 can include varying densities or dimensions. Moreover, the fluid permeable layer 572 can be manufactured according to various manufacturing methods, such as molding, extrusion, or sintering. The fluid permeable layer 572 may be substantially cylindrical in shape, and may fit within the fluid impermeable layer 568. In some embodiments, and majority of the outer surface 573 (shown in FIG. 5C) of the fluid permeable layer 572 interfaces with an inner surface 559 (shown in FIG. 5C) of the fluid impermeable layer 568.

The sheath 564 is configured to have a penis disposed therein. To facilitate fluid collection and improve comfort, the sheath 564 can be flexible thereby allowing the sheath 564 to correspond to the shape of a penis. For example, the flexible sheath 564 can at least partially collapse when the penis is flaccid and at least partially expand and bend to the shape of the penis as the penis becomes erect. Forming the layers of the sheath 564 from at least one of thin layers (e.g., less than 500 μm thick, and more particularly less than 250 μm thick, less than 100 μm thick, or less than 50 μm thick), flexible layers, or fabric can allow the sheath 564 to be sufficiently flexible.

The opening 510 is at least partially defined by the body 540 and extends from the first end 542 at least partially to the second end 544, thereby enabling a penis to enter the body 540. With a penis inserted into the opening 510 of the body 540, a male urethra is positioned within the fluid collection device 502 via the opening 510. The fluid collection device 502 is configured to receive the urine into the chamber 551 via the opening 510. For example, the opening 510 can exhibit an elongated shape that is configured to receive at least a portion of the penis, including the urethra. The opening 510 can exhibit an elongated shape to receive at least a portion of the penis, thereby only permitting the flow of the fluids along a path that corresponds to the elongated shape of the opening 510.

The ring 562 can be more rigid than the sheath 564. For example, the ring 562 can be formed from a flexible polymer that is at least one of thicker than the entire sheath 564 or exhibits a Young's modulus that is greater than sheath 564. As such, the ring 562 can provide some structure at or near the first end 542 of the body 540. The increased rigidity of the ring 562 can cause the first end 542 to remain open, thereby facilitating insertion of a penis into the urine collecting assembly 502. Further, in an embodiment, the increased rigidity of the ring 562 can enable the ring 562 to act as an attachment mechanism. For example, as illustrated, the ring 562 can include at least one protrusion 552 that extends from the rest of the body 540. In another example, the ring 562 can define a recess, include threads, or include any other attachment mechanism disclosed herein.

The sump 566 is configured to attach the rest of the urine collecting assembly 502 to the tubing 526. For example, the sump 566 can partially define an reservoir 575 extending through at least the fluid impermeable layer 568 thereby coupling the tubing 526 to the porous layer 572 and/or the opening 510. The tubing 526 can include an outlet and an inlet extending to or into the reservoir 575. Further, the sump 566 can close the second end 544 of the body 540. For example, the sump 566 can bunch up the sheath 542 and close any gaps that may form. The sump 566 can store fluids in the reservoir 575 therein. The reservoir 575 is an unoccupied portion of the sump 566 and is void of other material. In some embodiments, the reservoir 575 is defined at least partially by the fluid permeable layer 572 and the sump 566. During use, the fluids that are in the chamber 551 can flow through the fluid permeable layer 572 to the reservoir 575. The reservoir 575 can store at least some of the fluids therein and/or position the fluids for removal by the tubing 526. In some embodiments, at least a portion of the singular porous material of the fluid permeable layer 572 extends continuously between at least a portion of the opening 510 and the reservoir 575 to wick any fluid from the opening 510 directly to the reservoir 575.

The ring 562, the sheath 564, the sump 566, and the tubing 526 can be attached together using any suitable method. For example, at least two of the ring 562, the sheath 564, the sump 566, or the tubing 526 can be attached together using at least one of an interference fit, an adhesive, stitching, welding (e.g., ultrasonic welding), tape, any other suitable method, or combinations thereof.

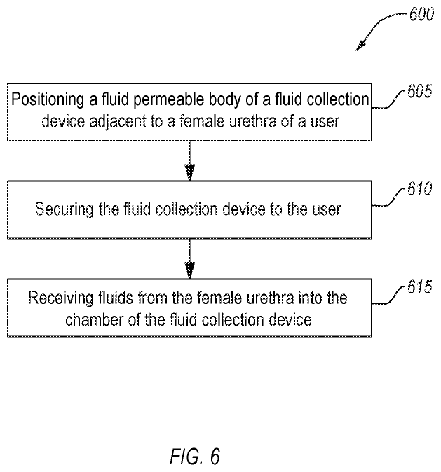

FIG. 6 is a flow diagram of a method 600 for collecting fluids. The method 600 includes an act 605 of positioning a fluid permeable body of a fluid collection device adjacent to a female urethra of a user. The fluid permeable body is disposed within a chamber of a fluid impermeable barrier of the fluid collection device and exposed to the female urethra of the user through an opening in the fluid collection device defined by the fluid impermeable barrier. The method 600 also includes an act 610 of securing the fluid collection device to the user. The method 600 also includes an act 615 of receiving fluids from the female urethra into the chamber of the fluid collection device. In some embodiments, the method 600 an act of applying suction effective to suction the fluids from the chamber via a conduit disposed therein.

While various aspects and embodiments have been disclosed herein, other aspects and embodiments are contemplated. The various aspects and embodiment disclosed herein are for purposes of illustration and are not intended to be limiting.

1. A fluid collection device, comprising:
a fluid permeable body consisting essentially of a singular porous hydrophilic polymer material in a cylindrical shape defining a bore extending at least partially therethrough;
and
a conduit positioned at least partially within the bore and including an inlet and an outlet,
wherein the singular porous hydrophilic polymer material extends substantially continuously from the conduit to an outer surface of the fluid permeable body,
wherein at least a portion of the outer surface of the fluid permeable body defines at least a portion of an outer surface of the fluid collection device configured to be placed adjacent to a urethra of a wearer, and at least the portion of the outer surface of the fluid collection device is free from coverage by gauze or other wicking material and a fluid impermeable barrier; and
wherein the singular porous hydrophilic polymer material of the fluid permeable body extends continuously from the conduit to at least the portion of the outer surface of the fluid permeable body that defines at least a portion of the outer surface of the fluid collection device.

2. The fluid collection device of claim 1, further comprising an antimicrobial material positioned on the fluid collection device where the fluid collection device contacts the wearer during use.

3. The fluid collection device of claim 1, wherein the fluid collection device is configured to bend or curve when positioned against the wearer.

4. The fluid collection device of claim 1, further comprising a reservoir of void space at a distal region of the fluid collection device, the reservoir being at least partially defined by the fluid permeable body.

5. The fluid collection device of claim 4, wherein the inlet of the conduit is positioned at a proximal end region of the reservoir.

6. The fluid collection device of claim 1, wherein:
the fluid permeable body includes a proximal body end and a distal body end;
the bore extends longitudinally through the fluid permeable body from the proximal body end to at least proximate to the distal body end.

7. The fluid collection device of claim 6, wherein the conduit extends into the bore through the proximal body end towards the distal body end.

8. The fluid collection device of claim 1, wherein the singular porous hydrophilic polymer material includes a polyolefin material.

9. A fluid collection system, comprising
a fluid storage container configured to hold a fluid;
a fluid collection device including a fluid permeable body consisting essentially of a singular porous hydrophilic polymer material in a cylindrical shape defining a bore extending at least partially therethrough, wherein at least a portion of an outer surface of the fluid permeable body defines at least a portion of an outer surface of the fluid collection device, and at least the portion of the outer surface of the fluid collection device is free from coverage by gauze or other wicking material and a fluid impermeable barrier, wherein the singular porous hydrophilic polymer material of the fluid permeable body extends continuously from the bore to at least the portion of the outer surface of the fluid permeable body that defines at least a portion of the outer surface of the fluid collection device, and wherein the fluid permeable body provides a latex-free single component of the fluid collection device that is free from the fluid impermeable barrier;
a conduit positioned at least partially within the bore of the fluid collection device and including an inlet and an outlet; and
a vacuum source fluidly coupled to one or more of the fluid storage container or the fluid collection device via the conduit, the vacuum source configured to draw fluid from the fluid collection device via the conduit.

10. The fluid collection system of claim 9, further comprising an antimicrobial material positioned on the fluid collection device where the fluid collection device contacts a wearer during use.

11. The fluid collection system of claim 9, wherein the fluid collection device is configured to bend or curve when positioned against a wearer.

12. The fluid collection system of claim 9, further comprising a reservoir of void space at a distal region of the fluid collection device, the reservoir being at least partially defined by the fluid permeable body.

13. The fluid collection system of claim 12, wherein the inlet of the conduit is positioned at a proximal end region of the reservoir.

14. The fluid collection system of claim 9, wherein:
the fluid permeable body includes a proximal body end and a distal body end;
the bore extends longitudinally through the fluid permeable body from the proximal body end to at least proximate to the distal body end.

15. The fluid collection system of claim 14, wherein the conduit extends into the bore through the proximal body end towards the distal body end.

16. The fluid collection system of claim 9, wherein the singular porous hydrophilic polymer material includes a polyolefin material.

17. A fluid collection device, comprising:
a fluid permeable body consisting essentially of a singular porous hydrophilic polymer material in a cylindrical shape defining a bore extending at least partially therethrough configured to receive a conduit;
wherein at least a portion of an outer surface of the fluid permeable body defines at least a portion of an outer surface of the fluid collection device, and at least the portion of the outer surface of the fluid collection device is free from coverage by gauze or other wicking material and a fluid impermeable barrier; and
wherein the singular porous hydrophilic polymer material of the fluid permeable body extends continuously from the bore to at least the portion of the outer surface of the fluid permeable body that defines at least a portion of the outer surface of the fluid collection device,
wherein the fluid permeable body provides a latex-free single component of the fluid collection device that is free from the fluid impermeable barrier.

18. The fluid collection device of claim 17, further comprising an antimicrobial material positioned on the fluid collection device where the fluid collection device contacts the wearer during use.

19. The fluid collection device of claim 17, further comprising a reservoir of void space at a distal region of the fluid collection device, the reservoir being at least partially defined by the fluid permeable body.

20. The fluid collection device of claim 17, wherein:
the fluid permeable body includes a proximal body end and a distal body end;
the bore extends longitudinally through the fluid permeable body from the proximal body end to at least proximate to the distal body end.
